(19) United States
(12) Patent Application Publication
(10) Pub. No.: US 2025/0033038 A1
(43) Pub. Date: Jan. 30, 2025

(54) MICROFLUIDIC DEVICES WITH ROUNDED CORNERS

(71) Applicant: QUREATOR, INC., San Diego, CA (US)

(72) Inventors: Jiho KIM, Seoul (KR); Kyusuk BAEK, San Diego, CA (US); Byungjun LEE, San Diego, CA (US); Tae Seon HWANG, San Diego, CA (US); Suryong KIM, Seoul (KR); Noo Li JEON, Seoul (KR)

(21) Appl. No.: 18/916,503

(22) Filed: Oct. 15, 2024

Related U.S. Application Data
(63) Continuation of application No. PCT/US2023/018862, filed on Apr. 17, 2023.
(60) Provisional application No. 63/331,652, filed on Apr. 15, 2022.

(51) Int. Cl. B01L 3/00

(52) U.S. Cl. CPC B01L 3/5027; B01L 2300/08

(57) ABSTRACT

A device includes a substrate, and one or more structures. At least a portion of a bottom surface of the one or more structures is coupled to the substrate, and the one or more structures include a first portion defining a first reservoir and a second portion defining a second reservoir that is in communication with the first reservoir via one or more channels defined at least between the substrate and the one or more structures. The second portion of the one or more structures does not include a non-rounded corner that extends continuously from the substrate to a top surface of the one or more structures.

### PRIORITY AND RELATED APPLICATIONS

This application is a continuation of PCT Patent Application No. PCT/US2023/018862, filed Apr. 17, 2023, entitled “Microfluidic Devices with Rounded Corners,” which claims priority to U.S. Provisional Patent Application No. 63/331,652 filed Apr. 15, 2022, entitled “Microfluidic Devices with Rounded Corners,” which is incorporated by reference herein in its entirety.

### TECHNICAL FIELD

This application relates to microfluidic devices and methods for using the same. In particular, this application relates to microfluidic devices with rounded corners for guiding transport of liquid within the microfluidic devices.

### SUMMARY

In accordance with some embodiments, a device includes a substrate having a top surface and a bottom surface opposite to the top surface of the substrate; and one or more structures having a top surface and a bottom surface opposite to the top surface of the one or more structures. At least a portion of the bottom surface of the one or more structures is coupled to the substrate. The one or more structures include a first portion defining a first reservoir and a second portion defining a second reservoir that is in communication with the first reservoir via one or more channels defined at least between the top surface of the substrate and the one or more structures. The second portion of the one or more structures does not include a non-rounded corner that extends continuously from the top surface of the substrate to the top surface of the one or more structures.

In accordance with some embodiments, a device includes a substrate having a top surface and a bottom surface opposite to the top surface of the substrate; and one or more structures having a top surface and a bottom surface opposite to the top surface of the one or more structures. At least a portion of the bottom surface of the one or more structures is coupled to the substrate. The one or more structures define a first reservoir and a second reservoir that is in communication with the first reservoir via one or more channels defined at least between the top surface of the substrate and the one or more structures. At least a first portion, of the one or more structures, defining the first reservoir does not include a non-rounded corner that extends continuously from the top surface of the substrate to the top surface of the one or more structures.

In accordance with some embodiments, a device includes a substrate; and one or more structures having a top surface and a bottom surface opposite to the top surface of the one or more structures. At least a portion of the bottom surface of the one or more structures is coupled to the substrate. The one or more structures include a first portion defining a first reservoir and a second portion defining a second reservoir that is in communication with the first reservoir via one or more channels defined at least between the substrate and the one or more structures. The second portion of the one or more structures does not include a non-rounded corner that extends continuously from the substrate to the top surface of the one or more structures.

In accordance with some embodiments, a device includes a substrate; and one or more structures having a top surface and a bottom surface opposite to the top surface of the one or more structures. At least a portion of the bottom surface of the one or more structures is coupled to the substrate. The one or more structures define a first reservoir and a second reservoir that is in communication with the first reservoir via one or more channels defined at least between the substrate and the one or more structures. At least a portion, of the one or more structures, defining the first reservoir does not include a non-rounded corner that extends continuously from the substrate to the top surface of the one or more structures.

In accordance with some embodiments, a method includes providing a first liquid to a first reservoir defined by a first portion of one or more structures having a top surface and a bottom surface opposite to the top surface of the one or more structures. At least a portion of the bottom surface of the one or more structures is coupled to a substrate having a top surface and a bottom surface opposite to the top surface of the substrate. The one or more structures also include a second portion defining a second reservoir that is in communication with the first reservoir via one or more channels defined at least between the top surface of the substrate and the one or more structures. At least the first portion of the one or more structures does not include a non-rounded corner that extends continuously from the top surface of the substrate to the top surface of the one or more structures. The method also includes providing a second liquid to the first reservoir.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1A illustrates components of a device in accordance with some embodiments.

FIG. 1B illustrates a top view of a body of the device in accordance with some embodiments.

FIG. 1C illustrates a cross-sectional view of the body of the device shown in FIG. 1B, in accordance with some embodiments.

FIG. 2A highlights a first portion of a second reservoir in accordance with some embodiments.

FIG. 2B highlights two ends of the first portion of the second reservoir in accordance with some embodiments.

FIG. 2C highlights a second portion of the second reservoir in accordance with some embodiments.

FIGS. 3A-3C illustrate rounded corners of a device in accordance with some embodiments.

FIGS. 4A-4D illustrate steps of providing liquids to a device in accordance with some embodiments.

FIGS. 5A-5C illustrate culturing one or more layers of tissue in accordance with some embodiments.

FIG. 6 shows an example image of an endothelial layer formed using a device described herein.

FIG. 7 shows an example image of a blood retinal barrier formed using a device described herein.

### DETAILED DESCRIPTION

Reference will now be made to embodiments, examples of which are illustrated in the accompanying drawings. In the following description, numerous specific details are set forth in order to provide an understanding of the various described embodiments. However, it will be apparent to one of ordinary skill in the art that the various described embodiments may be practiced without these specific details. In other instances, well-known methods, procedures, components, circuits, and networks have not been described in detail so as not to unnecessarily obscure aspects of the embodiments.

The “microfluidic channel” used herein refers to a path of fluid flow. In some cases, a fluid path defines a space in which cells or tissue is cultured and which is open at one or more sides (e.g., one lateral side, two lateral sides, three lateral sides, or four lateral sides) to be connected with another flow path or chamber so as to allow exchange of a culture medium and a fluid between adjacent fluids or chambers. In some embodiments, a microfluidic channel needs not be enclosed on two or more lateral sides.

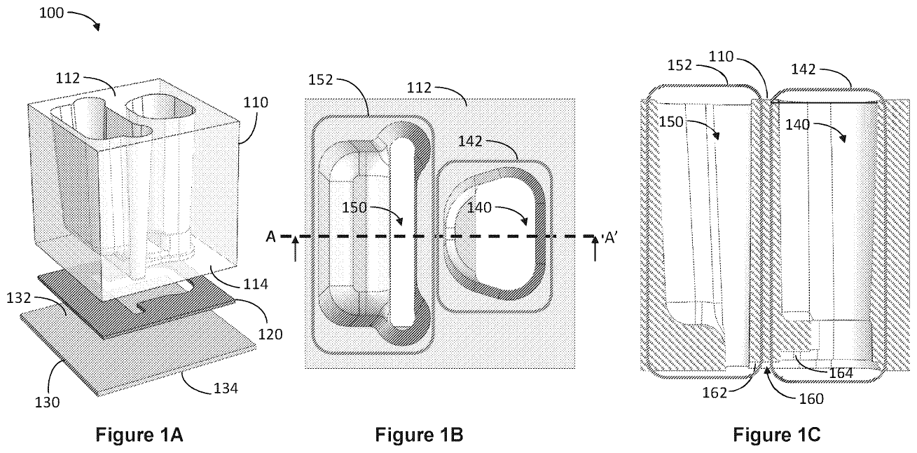

FIG. 1A illustrates components of a device 100 (in an exploded view) in accordance with some embodiments. In some embodiments, the device 100 is called a microfluidic device.

The device 100 includes a body 110 and a substrate 130. In some embodiments, the device 100 also includes a sealer 120.

In some embodiments, the body 110 has a top surface 112 and a bottom surface 114 that is opposite to the top surface 112. In some embodiments, the body 110 is made of a plastic material (e.g., polyethylene, polystyrene, polyvinyl chloride, polypropylene, polycarbonate, etc.). In some embodiments, the body 110 is made by using molding (e.g., injection molding, compression molding, insertion molding, etc.).

In some embodiments, the substrate 130 has a top surface 132 and a bottom surface 134 that is opposite to the top surface 132. In some embodiments, the substrate 130 is made of glass or a plastic material.

In some embodiments, the sealer 120 includes one or more adhesive layers.

In some embodiments, a method for making the device includes obtaining the body 110 and coupling (e.g., attaching) the body 110 to the substrate 130 to form a fluidic device (e.g., a device with one or more fluidic channels). In some embodiments, coupling the body 110 to the substrate 130 includes directly coupling the body 110 to the substrate 130 (e.g., by direct bonding) or indirectly coupling the body 110 to the substrate 130 (e.g., using the sealer 120). In some embodiments, coupling the body 110 to the substrate 130 includes attaching the body 110 to the sealer 120 and attaching the sealer 120 to the substrate 130 (or attaching the sealer 120 to the substrate 130 and attaching the body 110 to the sealer 120).

As shown in FIG. 1A, in some embodiments, a through-hole (or a cutout) is defined in the sealer 120 so that at least a portion of a liquid in the body 110 may contact directly with the substrate 130.

FIG. 1B illustrates a top view of the body 110 in accordance with some embodiments. In FIG. 1B, a first reservoir 140 and a second reservoir 150 defined by the body 110 are shown. In some embodiments, the first reservoir 140 is defined by a first portion 142 (e.g., a portion of the body 110 that substantially envelops the first reservoir 140) of the body 110. In some embodiments, the second reservoir 150 is defined by a second portion 152 (e.g., a portion of the body 110 that substantially envelops the second reservoir 140) of the body 110. In some embodiments, the first reservoir 140 and the second reservoir 150 are defined collectively by the body 110 and the substrate 130 (e.g., the substrate 130 defines a bottom of the first reservoir 140 and the second reservoir 150). In some embodiments, the first reservoir 140 and the second reservoir 150 are defined collectively by the body 110, the sealer 120, and the substrate 130 (e.g., depending on the thickness of the sealer 120, the sealer 120 may define one or more portions of side walls of the first reservoir 140 and the second reservoir 150). FIG. 1B also shows line AA′ from which the cross-sectional view of FIG. 1C is taken.

FIG. 1C illustrates a cross-sectional view of the body 110 shown in FIG. 1B, in accordance with some embodiments. FIG. 1C shows one or more channels 160 defined below the body 110. In FIG. 1C, the one or more channels 160 include a first channel 162 and a second channel 164. However, in some embodiments, the one or more channels 160 include additional channels (e.g., three or more channels) or fewer channels (e.g., one channel).

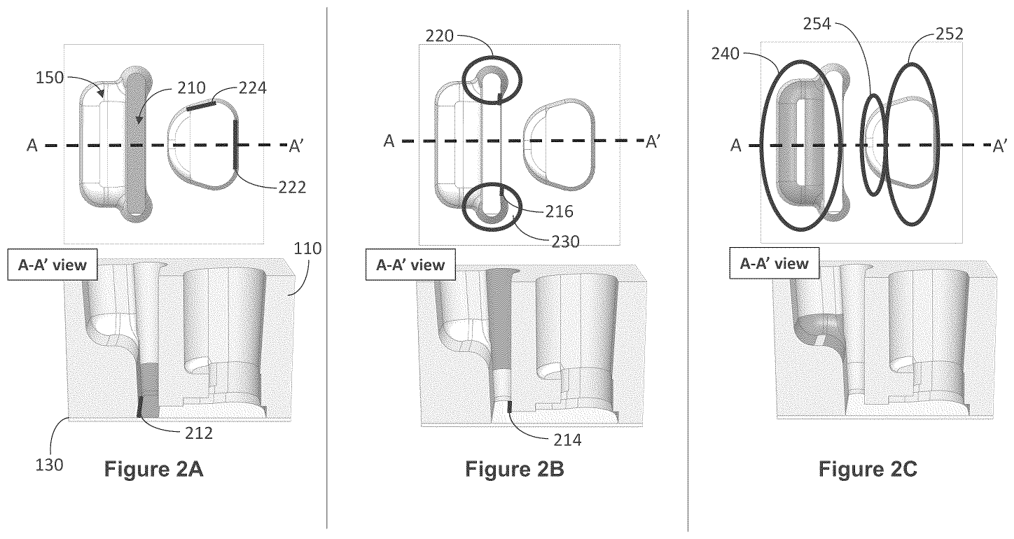

FIG. 2A highlights a first portion 210 of the second reservoir 150 in accordance with some embodiments.

In some embodiments, the first portion 210 of the second reservoir 150 includes a non-rounded corner 212. In some embodiments, the non-rounded corner 212 is defined by the body 110 and the substrate 130. In some embodiments, the non-rounded corner 212 is substantially parallel to the substrate 130 (e.g., the non-rounded corner 212 is located on a plane that is substantially parallel to the substrate 130). In some embodiments, the non-rounded corner 212 is in contact with (e.g., on) the substrate 130. In FIG. 2A, the non-rounded corner 212 defines one edge of a bottom portion of the second reservoir 150. The non-rounded corner 212 facilitates transport of a liquid along the non-rounded corner 212 (e.g., by capillary force).

As used herein, a rounded (or round) corner refers to a corner with a cross-section (e.g., a cross-section taken perpendicular to the corner) characterized by a radius of curvature equal to, or greater than, a predefined radius of curvature (e.g., 500 microns, 1 mm, 1.5 mm, 2 mm, 2.5 mm, 3 mm, 3.5 mm, 4 mm, 4.5 mm, 5 mm, etc.). For example, a rounded corner may be formed by filleting a non-rounded corner (or by molding a corner with a certain radius of curvature equal to, or greater than, the predefined radius of curvature). In some embodiments, the rounded corner is a rounded concave corner. As used herein, a non-rounded corner refers to a corner that is not a rounded corner as defined herein. An example of a non-rounded corner is a corner or edge defined by two planes intersecting, and perpendicular to, each other with a cross-section having a sharp 90° angle with no rounding. Other examples of a non-rounded corner include a corner or edge defined by two non-parallel intersecting planes, such as a corner having a cross-section with an acute or obtuse angle and without rounding. Another example of a non-rounded corner is a corner with a cross-section characterized by a radius of curvature less than the predefined radius of curvature (e.g., a radius of curvature less than 500 microns, 400 microns, 300 microns, 200 microns, 100 microns, 90 microns, 80 microns, 70 microns, 60 microns, 50 microns, 40 microns, 30 microns, 20 microns, 10 microns, etc.).

FIG. 2A also indicates that at least a portion of an edge, opposite to the rounded corner 212, of the bottom portion of the second reservoir 150 remains open (e.g., due to the connection to the one or more channels 160). Thus, a liquid provided into the second reservoir 150 may not flow along the edge opposite to the rounded corner 212 to meet with the liquid transported along the rounded corner 212. This may reduce the formation or trapping of bubbles, which may occur when the liquid flowing in opposite directions meet.

FIG. 2B highlights two ends 220 and 230 of the first portion of the second reservoir in accordance with some embodiments. In some embodiments, at least one of the two ends 220 and 230 of the first portion of the second reservoir has a shape of at least a portion of a cone (or a cylinder). This shape allows insertion and removal of a liquid dispensing apparatus, such as a pipette tip, reducing, or eliminating, an additional air pressure applied to the liquid in the second reservoir, which increases the uniformity in distribution of cells provided to the second reservoir for cell culture. In some embodiments, each of the two ends 220 and 230 of the first portion of the second reservoir has a shape of at least a portion of a cone (or a cylinder).

FIG. 2C highlights a second portion 240 of the second reservoir in accordance with some embodiments. The second portion 240 of the second reservoir increases the capacity of the second reservoir, allowing the second reservoir to retain a large volume of liquid for cell culturing and/or tissue formation. In some embodiments, the second portion 240 of the second reservoir does not include any non-rounded corner.

In some embodiments, the device includes one or more non-rounded convex corners that extend continuously from the substrate to the top surface of the body (e.g., outside edges of the body that extend from the substrate to the top surface of the body).

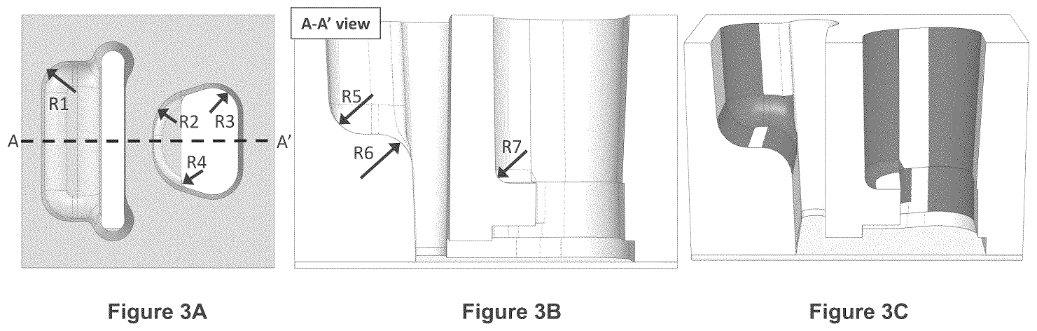

FIGS. 3A-3C illustrate rounded corners of a device in accordance with some embodiments. As shown in FIGS. 3A and 3B, the radii of curvature for different corners may be represented by R1, R2, R3, R4, R5, R6, and R7. The radii of curvature, such as R1, R2, R3, and R4, for corners that substantially extend vertically (e.g., substantially perpendicular to the substrate) are selected to reduce or prevent vertical transport of a liquid (e.g., upward transport by capillary force). The radii of curvature, such as R5 and R7, for corners that substantially extend horizontally (e.g., substantially parallel to the substrate) are selected to reduce or prevent horizontal transport of a liquid (e.g., a lateral transport by capillary force). The radius of curvature R6 is selected to reduce or prevent bubble pinning at the end of the second reservoir. In some embodiments, R1, R2, R3, R4, R5, R6, and R7 represent a same radius of curvature (e.g., R1, R2, R3, R4, R5, R6, and R7 have a same value). In some embodiments, two or more selected from R1, R2, R3, R4, R5, R6, or R7 have a same value. In some embodiments, two or more selected from R1, R2, R3, R4, R5, R6, or R7 have different values. In some embodiments, R1, R2, R3, R4, R5, R6, and R7 have different values.

FIG. 3C shows corners characterized by the radii of curvature R1, R2, R3, R4, R5, R6, and R7 highlighted with shading.

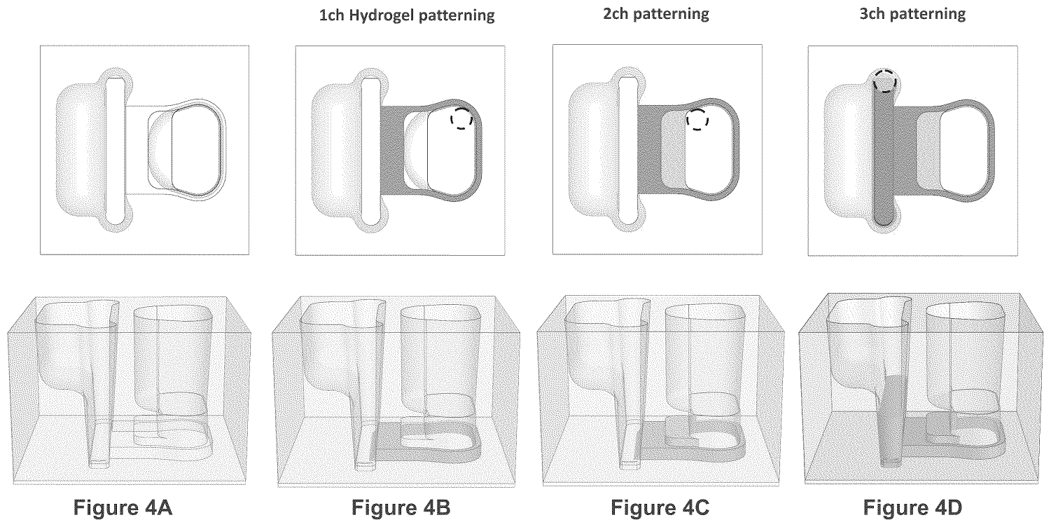

FIGS. 4A-4D illustrate steps of providing liquids to a device in accordance with some embodiments.

FIG. 4A shows a device (e.g., the device 100 described with respect to FIGS. 1A-1C, 2A-2C, and 3A-3C).

FIG. 4B illustrates providing a first liquid to the first reservoir 140 at a location away from the one or more channels. The first liquid travels along a non-rounded corner to (and fills) a portion of the one or more channels (e.g., the channel 162).

FIG. 4C illustrates providing a second liquid to the first reservoir 140 at a location adjacent to the one or more channels. The second liquid travels along a pattern of the first liquid to (and fills) a portion of the one or more channels (e.g., the channel 164).

FIG. 4D illustrates providing a third liquid to the second reservoir 150. The third liquid fills at least a portion of the second reservoir 150 and comes into contact with a portion of the first liquid (in the one or more channels).

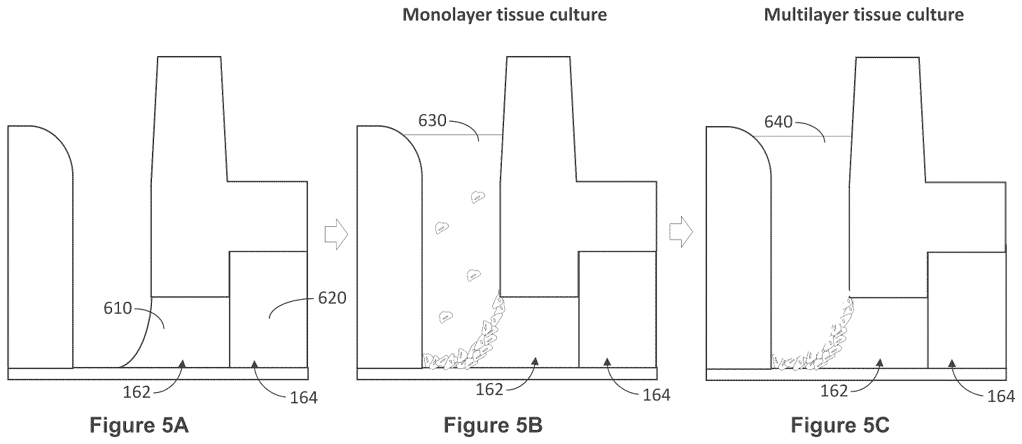

FIGS. 5A-5C illustrate culturing one or more layers of tissue in accordance with some embodiments. FIGS. 5A-5C show an enlarged cross-section of a device (e.g., the device 100 described with respect to FIGS. 1A-1C, 2A-2C, and 3A-3C) with patterned liquids (e.g., as described with respect to FIGS. 4B-4D).

For example, FIG. 5A shows the device with the first liquid 610 (e.g., hydrogel) in the first channel 162, and the second liquid 620 (e.g., a cell suspension or a mixture of cells and hydrogel). This forms a meniscus for a layered tissue. In some cases, the volume of the hydrogel determines the shape of the meniscus. Thus, changing the volume of the hydrogel changes the shape of the meniscus (e.g., a first volume of the hydrogel is provided at a first time to form a hydrogel layer having a first shape adjacent to the first channel 162 and a second volume of the hydrogel is provided at a second time distinct from the first time to form a hydrogel layer having a second shape distinct from the first shape).

FIG. 5B shows the device with the third liquid 630 (e.g., a cell suspension) in the second reservoir. After culturing, a tissue layer matures (e.g., a first layer of the cells is formed on the meniscus of the hydrogel).

FIG. 5C shows the device with a fourth liquid 640 (e.g., a cell suspension) in the second reservoir. In some embodiments, at least a portion of the third liquid 630 is removed from the second reservoir and then the fourth liquid 640 is provided to the second reservoir. After culturing, a second layer of the cells is formed over the first layer of the cells. Thus, this allows formation of a multilayer tissue. In some embodiments, the third liquid and the fourth liquid are identical (e.g., the third liquid and the fourth liquid contain the same type of cells). In some embodiments, the fourth liquid is distinct from the third liquid (e.g., the third liquid and the fourth liquid contain different types of cells, or the third liquid and the fourth liquid contain the same type of cells at different concentrations).

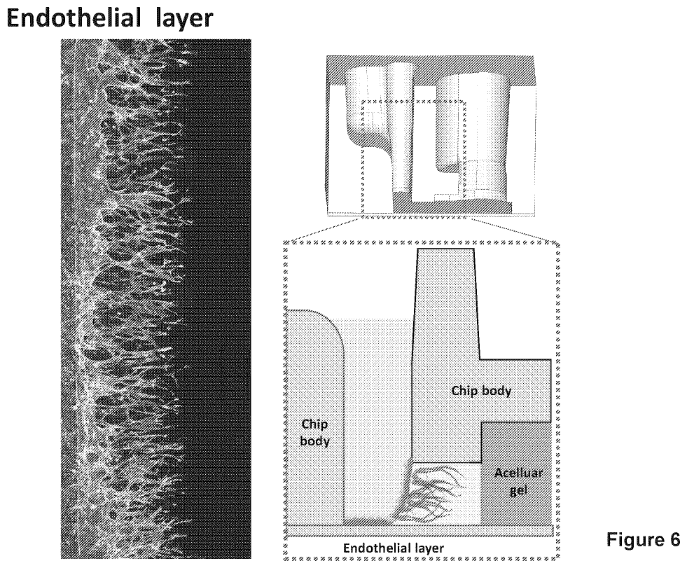

FIG. 6 shows an example image of an endothelial layer formed using a device described herein (e.g., the device 100 described with respect to FIGS. 1A-1C, 2A-2C, and 3A-3C). FIG. 6 also shows angiogenesis from the endothelial layer. An example protocol for angiogenesis is as follows:

### Example Protocol 1—Angiogenesis

### 1) Cell Preparation

HUVECs (Lonza) were cultured in endothelial growth medium 2 (EGM-2, Lonza). The cells were incubated at 37° C. in 5% CO2 for three days prior to loading in the devices. Cultured HUVECs were removed from the culture dish using 0.25% Trypsin-EDTA (Hyclone). HUVECs were then re-suspended in the EGM-2 solution.

### 2) Cell Seeding and Culture in the Device

Before starting patterning, hydrophilic treatment was done to improve hydrophilicity of the surface of the device.

After mixing the cell-free fibrinogen solution with thrombin (Sigma), drop the solution to one of the inner wedges formed by the bottom and sidewalls of the device.

A small amount of hydrogel moves along the hydrophilic wedge from the dropping position, and the hydrogel selectively fills the lowest height channel in the middle.

After first patterning, wait for hydrogel gelation. As a result, the gel patterned in the central channel makes the open-top channel and the open-side channel into two separate regions so that the next fluid can be loaded.

For the second patterning, mix cell-free fibrinogen solution with thrombin (Sigma) and load the mixed solution with a pipette placed near the open-side channel.

After gelation of second patterned hydrogel, dispense cell suspension containing HUVECs with a pipette into the partially open inlet.

Only fill the reservoir with an open-side channel of EGM-2 medium to generate interstitial flow using medium height difference.

Incubate it in an incubator for 18 hours at 37° C. and 5% CO2.

Then, remove the medium from both reservoirs. In the case of removing the medium of the open-top channel side reservoir, suction is performed on the flat part beside the top of the open-top channel, not to damage the tissue layer cultured inside the channel. For the reservoir on the open-side channel, remove the medium from the bottom in the center of the reservoir.

Dispense fresh EGM-2 medium containing angiogenic factors on the reservoir with an open-side channel at the same position where removal has been done.

Refresh the medium every day in the same process until 4 days of culture.

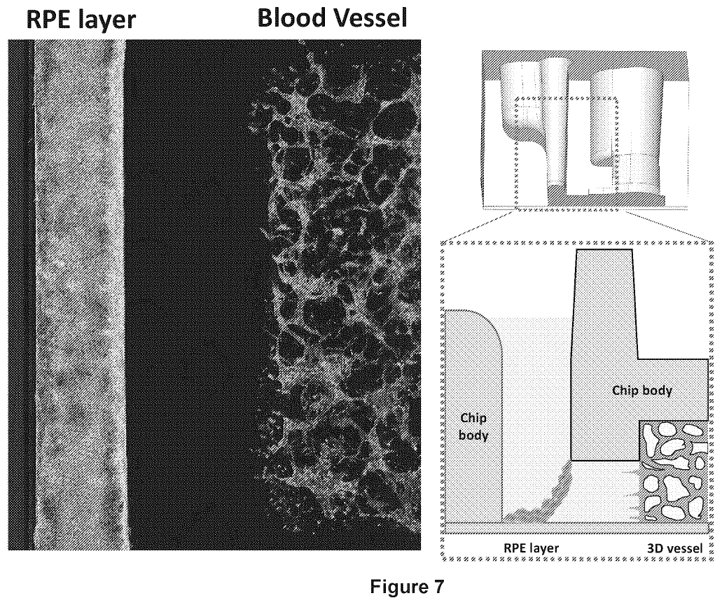

FIG. 7 shows an example image of a blood retinal barrier formed using a device described herein (e.g., the device 100 described with respect to FIGS. 1A-1C, 2A-2C, and 3A-3C). In particular, FIG. 7 shows a layer of retinal pigment epithelium (RPE) formed on the meniscus of the hydrogel and blood vessels formed in the second channel. An example protocol for forming the blood retinal barrier is as follows:

### Example Protocol 2—Blood Retinal Barrier

### 1) Cell Preparation

HUVECs (Lonza) were cultured in endothelial growth medium 2 (EGM-2, Lonza). human derived retinal pigment epithelium (RPE) cells were cultured in RPE culture medium. The cells were incubated at 37° C. in 5% CO2 for three days prior to loading in the devices. Cultured HUVECs and RPE cells were removed from the culture dish using 0.25% Trypsin-EDTA (Hyclone). HUVECs were then re-suspended in a bovine fibrinogen solution. RPE cells were re-suspended in a RPE culture medium.

### 2) Cell Seeding and Culture in the Device

Before starting patterning, hydrophilic treatment was done to improve hydrophilicity of the surface of the device.

After mixing the cell-free fibrinogen solution with thrombin (Sigma), drop the solution to one of the inner wedges formed by the bottom and sidewalls of the device.

A small amount of hydrogel moves along the hydrophilic wedge from the dropping position, and the hydrogel selectively fills the lowest height channel in the middle.

After first patterning, wait for hydrogel gelation. As a result, the gel patterned in the central channel makes the open-top channel and the open-side channel into two separate regions so that the next fluid can be loaded.

For the second patterning, mix fibrinogen solution containing HUVECs with thrombin (Sigma) and load the mixture with a pipette placed near the open-side channel.

After gelation of second patterned hydrogel, dispense RPE medium with a pipette fixed in the partially open inlet.

Fill both reservoir and incubate it in an incubator for 18 hours at 37° C. and 5% CO2.

Remove the medium from both reservoirs. In the case of removing the medium of the open-top channel side reservoir, suction is performed on the flat part beside the top of the open-top channel, not to damage the tissue layer cultured inside the channel. Then, Insert the pipette into the partially opened inlet and remove the remaining medium in the open-top channel.

For the reservoir on the open-side channel, remove the medium from the bottom in the center of the reservoir.

Dispense cell suspension containing RPEs with a pipette into the partially open inlet.

Fill the HUVEC side reservoir with EGM-2 and RPE side reservoir with RPE culture medium.

Refresh the medium once every 2 days with the medium suitable for each cell type until 6 days of culture.

In view of these principles and examples, we turn to certain embodiments.

In accordance with some embodiments, a device (e.g., the device 100) includes a substrate (e.g., substrate 130) having a top surface (e.g., top surface 132) and a bottom surface (e.g., bottom surface 134) opposite to the top surface of the substrate. The device also includes one or more structures (e.g., body 110) having a top surface (e.g., top surface 112) and a bottom surface (e.g., bottom surface 114) opposite to the top surface of the one or more structures. At least a portion of the bottom surface of the one or more structures is coupled to the substrate (e.g., the bottom surface of the one or more structures is coupled to the substrate directly or indirectly). The one or more structures include a first portion (e.g., a portion of the body 110 surrounding the first reservoir 140) defining a first reservoir (e.g., the first reservoir 140) and a second portion (e.g., a portion of the body 110 surrounding the second reservoir 150) defining a second reservoir (e.g., the second reservoir 150) that is in communication (e.g., fluidic communication) with the first reservoir via one or more channels (e.g., one or more channels 160) defined at least between the top surface of the substrate and the one or more structures. For example, a liquid in the first reservoir may flow to the second reservoir via the one or more channels in the absence of surface tension. The second portion of the one or more structures (e.g., defining the second reservoir) does not include a non-rounded corner (e.g., a non-rounded concave corner) that extends continuously from the top surface of the substrate to the top surface of the one or more structures. For example, the second reservoir does not have a non-rounded (e.g., sharp) corner that extends continuously from the substrate to the top surface of the one or more structures.

However, in some embodiments, the second reservoir may have a non-rounded corner that does not extend continuously from the substrate to the top surface of the one or more structures. For example, in some embodiments, the second portion of the one or more structures includes a first non-rounded corner (e.g., the non-rounded corner 212) that extends substantially parallel along the top surface of the substrate.

In some embodiments, the first non-rounded corner is located away from the one or more channels (e.g., FIG. 2A).

In some embodiments, the second portion of the one or more structures does not include a non-rounded corner that extends from the top surface of the substrate. For example, in some embodiments, the second portion of the one or more structures includes a second non-rounded corner that extends from the top surface of the substrate without extending continuously to the top surface of the one or more structures.

In some embodiments, the second portion of the one or more structures does not include a non-rounded corner that extends to the top surface of the one or more structures. For example, in some embodiments, the second portion of the one or more structures includes a third non-rounded corner that extends to the top surface of the one or more structures without extending continuously from the top surface of the substrate.

In some embodiments, the second portion of the one or more structures has a corner, one or more portions of which are non-rounded, but the one or more non-rounded portions do not extend continuously from the substrate to the top surface of the one or more structures.

In some embodiments, the second reservoir has a first portion with a first end (e.g., end 220) and a second end (e.g., end 230) separated from the first end.

In some embodiments, the one or more structures and the substrate define a non-rounded corner (e.g., the non-rounded corner 212) that extends from the first end of the second reservoir to the second end of the second reservoir.

In some embodiments, the first portion of the second reservoir extends between the first end and the second end along the one or more channels (e.g., an open side opposite to the non-rounded corner that connects to the one or more channels).

In some embodiments, the first portion of the second reservoir is positioned adjacent to the one or more channels so that the first portion of the second reservoir is in communication with the one or more channels (e.g., FIG. 2A).

In some embodiments, the one or more structures and the substrate define a non-rounded corner (e.g., a non-rounded corner 214) that extends from the first end of the second reservoir to the one or more channels.

In some embodiments, the one or more structures and the substrate define a non-rounded corner (e.g., a non-rounded corner 216) that extends from the second end of the second reservoir to the one or more channels.

In some embodiments, the first end includes a partially enclosed conical shape.

In some embodiments, the second end includes a partially enclosed conical shape.

In some embodiments, the second reservoir includes a second portion (e.g., the second portion 240). The first portion of the second reservoir is characterized by a first depth, and the second portion of the second reservoir is characterized by a second depth that is less than the first depth.

In some embodiments, the second portion of the second reservoir is positioned away from the one or more channels (e.g., FIG. 2C).

In some embodiments, the second portion of the second reservoir does not include a non-rounded corner (e.g., FIG. 2C).

In some embodiments, the first portion of the one or more structures does not include a non-rounded corner that extends continuously from the top surface of the substrate to the top surface of the one or more structures. For example, the first reservoir does not have a non-rounded (e.g., sharp) corner that extends continuously from the substrate to the top surface of the one or more structures.

However, in some embodiments, the first reservoir may have a non-rounded corner that does not extend continuously from the substrate to the top surface of the one or more structures. For example, in some embodiments, the first portion of the one or more structures includes a fourth non-rounded corner (e.g., the non-rounded corner 222) that extends substantially parallel along the top surface of the substrate.

In some embodiments, the fourth non-rounded corner is located away from the one or more channels (e.g., FIG. 2A).

In some embodiments, the first portion of the one or more structures includes a fifth non-rounded corner (e.g., the non-rounded corner 224) that extends (e.g., directly or indirectly) from the fourth non-rounded corner to the one or more channels.

In some embodiments, the first portion of the one or more structures does not include a non-rounded corner that extends from the top surface of the substrate. For example, in some embodiments, the first portion of the one or more structures includes a sixth non-rounded corner that extends from the top surface of the substrate without extending continuously to the top surface of the one or more structures.

In some embodiments, the first portion of the one or more structures does not include a non-rounded corner that extends to the top surface of the one or more structures. For example, in some embodiments, the first portion of the one or more structures includes a seventh non-rounded corner that extends to the top surface of the one or more structures without extending continuously from the top surface of the substrate.

In some embodiments, a first portion (e.g., the first portion 252) of the first reservoir extends along the one or more channels.

In some embodiments, the first portion of the first reservoir is positioned adjacent to (or extends to) the one or more channels so that the first portion of the first reservoir is in communication (e.g., fluidic communication) with the one or more channels. For example, a liquid in the first reservoir may flow into the one or more channels.

In some embodiments, the first reservoir includes a second portion (e.g., the second portion 254). The first portion of the first reservoir is characterized by a third depth, and the second portion of the first reservoir is characterized by a fourth depth that is less than the third depth.

In some embodiments, the first portion of the first reservoir extends under the second portion of the first reservoir to the one or more channels (e.g., FIG. 2C).

In some embodiments, the second portion of the first reservoir is positioned toward the one or more channels (e.g., FIG. 2C).

In some embodiments, the second portion of the first reservoir does not include a non-rounded corner.

In accordance with some embodiments, a device includes a substrate having a top surface and a bottom surface opposite to the top surface of the substrate; and one or more structures having a top surface and a bottom surface opposite to the top surface of the one or more structures. At least a portion of the bottom surface of the one or more structures is coupled to the substrate. The one or more structures define a first reservoir and a second reservoir that is in communication with the first reservoir via one or more channels defined at least between the top surface of the substrate and the one or more structures. At least a first portion, of the one or more structures, defining the first reservoir does not include a non-rounded corner (e.g., a non-rounded concave corner) that extends continuously from the top surface of the substrate to the top surface of the one or more structures.

In some embodiments, the first portion of the one or more structures includes a fourth non-rounded corner that extends substantially parallel along the top surface of the substrate.

In some embodiments, the fourth non-rounded corner is located away from the one or more channels.

In some embodiments, the first portion of the one or more structures includes a fifth non-rounded corner that extends from the fourth non-rounded corner to the one or more channels.

In some embodiments, the first portion of the one or more structures does not include a non-rounded corner that extends from the top surface of the substrate.

In some embodiments, the first portion of the one or more structures includes a sixth non-rounded corner that extends from the top surface of the substrate without extending continuously to the top surface of the one or more structures.

In some embodiments, the first portion of the one or more structures does not include a non-rounded corner that extends to the top surface of the one or more structures.

In some embodiments, the first portion of the one or more structures includes a seventh non-rounded corner that extends to the top surface of the one or more structures without extending continuously from the top surface of the substrate.

In some embodiments, a first portion of the first reservoir extends along the one or more channels.

In some embodiments, the first portion of the first reservoir is positioned adjacent to the one or more channels so that the first portion of the first reservoir is in communication with the one or more channels.

In some embodiments, the first reservoir includes a second portion. The first portion of the first reservoir is characterized by a third depth; and the second portion of the first reservoir is characterized by a fourth depth that is less than the third depth.

In some embodiments, the second portion of the first reservoir is positioned toward the one or more channels.

In some embodiments, the second portion of the first reservoir does not include a non-rounded corner.

In some embodiments, the entire surfaces of the first reservoir and the second reservoir are hydrophilic.

In accordance with some embodiments, a method includes providing a first liquid to a first reservoir (e.g., FIG. 4B) defined by a first portion of one or more structures having a top surface and a bottom surface opposite to the top surface of the one or more structures. At least a portion of the bottom surface of the one or more structures is coupled to a substrate having a top surface and a bottom surface opposite to the top surface of the substrate. The one or more structures also include a second portion defining a second reservoir that is in communication with the first reservoir via one or more channels defined at least between the top surface of the substrate and the one or more structures. At least the first portion of the one or more structures does not include a non-rounded corner that extends continuously from the top surface of the substrate to the top surface of the one or more structures.

The method also includes providing a second liquid to the first reservoir (e.g., FIG. 4C).

In some embodiments, the first liquid is provided to the first reservoir at a location away from the one or more channels (e.g., FIG. 4B).

In some embodiments, the first portion of the one or more structures includes a first non-rounded corner that extends substantially parallel along the top surface of the substrate. The first liquid is provided adjacent to the first non-rounded corner so that the first liquid is transported along at least a portion of the first non-rounded corner toward the one or more channels.

In some embodiments, the first non-rounded corner is located away from the one or more channels.

In some embodiments, the first portion of the one or more structures includes a second non-rounded corner that extends from the first non-rounded corner to the one or more channels so that the first liquid is transported from the first non-rounded corner to the one or more channels along the second non-rounded corner.

In some embodiments, the one or more channels includes a first channel having a first height and a second channel having a second height greater than the first height.

In some embodiments, the first liquid substantially fills the first channel, and the second liquid substantially fills the second channel.

In some embodiments, the first channel is located adjacent to the second reservoir, and the second channel is located adjacent to the first reservoir.

In some embodiments, the second liquid is provided to the first reservoir at a location adjacent to the one or more channels.

In some embodiments, the method includes providing a third liquid to the second reservoir.

In some embodiments, the second reservoir has a first portion with a first end and a second end separated from the first end, and the first portion of the second reservoir extends between the first end and the second end along the one or more channels.

In some embodiments, the third liquid is provided to the first end of the first portion of the second reservoir.

In some embodiments, the second portion of the one or more structures includes a third non-rounded corner that extends substantially parallel along the top surface of the substrate.

In some embodiments, the third non-rounded corner is located away from the one or more channels.

In some embodiments, the third liquid is transported from the first end of the first portion of the second reservoir to the second end of the first portion of the second reservoir along the third non-rounded corner.

In some embodiments, the second liquid provided to the first reservoir is characterized by a first liquid height, and the third liquid provided to the second reservoir is characterized by a second liquid height that is distinct from the first liquid height.

In some embodiments, the method includes providing a fourth liquid to the first reservoir so that the fourth liquid in the first reservoir is characterized by a third liquid height. The method also includes providing a fifth liquid to the second reservoir so that the fifth liquid in the second reservoir is characterized by a fourth liquid height that is distinct from the third liquid height.

In accordance with some embodiments, a device includes a substrate and one or more structures having a top surface and a bottom surface opposite to the top surface of the one or more structures. At least a portion of the bottom surface of the one or more structures is coupled to the substrate. The one or more structures include a first portion defining a first reservoir and a second portion defining a second reservoir that is in communication with the first reservoir via one or more channels defined at least between the substrate and the one or more structures. The second portion of the one or more structures does not include a non-rounded corner that extends continuously from the substrate to the top surface of the one or more structures.

In accordance with some embodiments, a device includes a substrate and one or more structures having a top surface and a bottom surface opposite to the top surface of the one or more structures. At least a portion of the bottom surface of the one or more structures is coupled to the substrate. The one or more structures define a first reservoir and a second reservoir that is in communication with the first reservoir via one or more channels defined at least between the substrate and the one or more structures. At least a portion, of the one or more structures, defining the first reservoir does not include a non-rounded corner that extends continuously from the substrate to the top surface of the one or more structures.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. The embodiments were chosen and described in order to best explain the principles of the various described embodiments and their practical applications, to thereby enable others skilled in the art to best utilize the principles and the various described embodiments with various modifications as are suited to the particular use contemplated.

What is claimed is:

1. A device, comprising:
a substrate having a top surface and a bottom surface opposite to the top surface of the substrate; and
one or more structures having a top surface and a bottom surface opposite to the top surface of the one or more structures, at least a portion of the bottom surface of the one or more structures coupled to the substrate, the one or more structures including a first portion defining a first reservoir and a second portion defining a second reservoir that is in communication with the first reservoir via one or more channels defined at least between the top surface of the substrate and the one or more structures, wherein the second portion of the one or more structures does not include a non-rounded corner that extends continuously from the top surface of the substrate to the top surface of the one or more structures.

2. The device of claim 1, wherein:
the second portion of the one or more structures includes a first non-rounded corner that extends substantially parallel along the top surface of the substrate.

3. The device of claim 2, wherein:
the first non-rounded corner is located away from the one or more channels.

4. The device of claim 1, wherein:
the second portion of the one or more structures does not include a non-rounded corner that extends from the top surface of the substrate.

5. The device of claim 1, wherein:
the second portion of the one or more structures includes a second non-rounded corner that extends from the top surface of the substrate without extending continuously to the top surface of the one or more structures.

6. The device of claim 1, wherein:
the second portion of the one or more structures does not include a non-rounded corner that extends to the top surface of the one or more structures.

7. The device of claim 1, wherein:
the second portion of the one or more structures includes a third non-rounded corner that extends to the top surface of the one or more structures without extending continuously from the top surface of the substrate.

8. The device of claim 1, wherein:
the second reservoir has a first portion with a first end and a second end separated from the first end.

9. The device of claim 8, wherein:
the one or more structures and the substrate define a non-rounded corner that extends from the first end of the second reservoir to the second end of the second reservoir.

10. The device of claim 8, wherein:
the first portion of the second reservoir extends between the first end and the second end along the one or more channels.

11. The device of claim 8, wherein:
the first portion of the second reservoir is positioned adjacent to the one or more channels so that the first portion of the second reservoir is in communication with the one or more channels.

12. The device of claim 8, wherein:
the one or more structures and the substrate define a non-rounded corner that extends from the first end of the second reservoir to the one or more channels.

13. The device of claim 8, wherein:
the one or more structures and the substrate define a non-rounded corner that extends from the second end of the second reservoir to the one or more channels.

14. The device of claim 8, wherein:
the first end includes a partially enclosed conical shape.

15. The device of claim 8, wherein:
the second end includes a partially enclosed conical shape.

16. The device of claim 8, wherein:
the second reservoir includes a second portion;
the first portion of the second reservoir is characterized by a first depth; and
the second portion of the second reservoir is characterized by a second depth that is less than the first depth.

17. The device of claim 16, wherein:
the second portion of the second reservoir is positioned away from the one or more channels.

18. The device of claim 16, wherein:
the second portion of the second reservoir does not include a non-rounded corner.

19. The device of claim 1, wherein:
the first portion of the one or more structures does not include a non-rounded corner that extends continuously from the top surface of the substrate to the top surface of the one or more structures.

20. The device of claim 19, wherein:
the first portion of the one or more structures includes a fourth non-rounded corner that extends substantially parallel along the top surface of the substrate.
